(19) United States
(12) Patent Application Publication
(10) Pub. No.: US 2020/0029975 A1
(43) Pub. Date: Jan. 30, 2020

(54) AUTOMATIC TOURNIQUET FOR EMERGENCY OR SURGERY

(71) Applicant: KONINKLIJKE PHILIPS N.V., EINDHOVEN (NL)

(72) Inventors: ADAM JONES, EINDHOVEN (NL); KIRAN HAMILTON J. DELLIMORE, UT (NL)

(21) Appl. No.: 16/469,239

(22) PCT Filed: Dec. 12, 2017
(86) PCT No.: PCT/EP2017/082447

§ 371 Date: Jun. 13, 2019

Related U.S. Application Data
(60) Provisional application No. 62/433,481, filed on Dec. 13, 2016.

(51) Int. Cl. A61B 17/135

(52) U.S. Cl. CPC A61B 17/1355; A61B 2090/061

(57) ABSTRACT

An inflatable tourniquet system for arterial blood occlusion of a leg or arm, e.g. after injury or for surgery. A tourniquet (TQ) is to be manually fastened around the limb by a user, e.g. a first aid helper, e.g. an untrained person. A manual inflator (B) is used to inflatable the tourniquet to apply pressure for occlusion of arterial blood flow to the limb. An electric circuit (CC) measures an electrical input from a length sensor (C), e.g. an electric conductor, and to determine a value (R), e.g. electric resistance, indicative of circumference of the limb accordingly, when the tourniquet has been fastened around the limb. A blood pressure measuring circuit (BP) automatically determines a systolic blood pressure (SBP) in response to input from a pressure sensor (PS) arranged to measure a pressure (PR) of the tourniquet. A processor (P) is programmed to operate according to a control algorithm which calculates a target pressure (AOP, OAOP) in response to the measured SBP, and the value (R) indicative of circumference of the limb. Then, the processor monitors input from the pressure sensor (PS) and compares the sensed pressure with the calculated target pressure (AOP, OAOP). Visual and/or audible feedback (FB) is give to the user, when the pressure (PR) of the tourniquet (TQ) is within an interval of the target pressure (AOP, OAOP). In some embodiments, the manual inflator (B) process may be used to provide energy harvesting for electric powering the system.

### FIELD OF THE INVENTION

The invention relates to medical devices. Specifically, to the field of tourniquet devices with the aim of reducing blood flow to a limb of a person or an animal. Especially tourniquet devices for preventing hemorrhage due to injuries or for reducing blood flow during surgery. More specifically, the invention provides a method and a tourniquet device for automatic blood occlusion of limb.

### BACKGROUND OF THE INVENTION

The most common cause of preventable deaths both on the battlefield and on the streets of the U.S. is uncontrolled hemorrhage from a limb. Tourniquets are effective devices in the treatment of blast wounds, penetrating trauma (such as stab wounds or gunshots), industrial accidents and injuries sustained in remote, resource scarce environments, where more comprehensive medical attention is not immediately available.

However, the use of tourniquets in prehospital settings is widely debated. There is extensive evidence to support the negative consequences associated with the inappropriate or prolonged use of tourniquets, including skin and nerve damage, pain, blood clots and tissue death. The damage caused by the pressure of a tourniquet corresponds to both the pressure applied and the duration of its application. Emergency tourniquets have been recognized as crucial life-saving tools in disaster response scenarios, but are difficult to apply correctly without prior training and can cause severe damage if not properly applied.

In both the US and the UK, efforts are being made to equip emergency services personnel with effective, commercially produced tourniquets and to provide training in their proper application. It is important that emergency tourniquets are available for quick and effective use in public settings where there is a risk of mass injury, however, the greatest obstacle is the lack of knowledge and training of the general public, which is essential if a tourniquet is to be safely applied to an injured person. For this reason, tourniquets are not widely included in emergency first aid kits provided in public spaces. The tourniquet must have sufficient pressure applied to ensure that bleeding is fully controlled, while minimising the risk of damage caused by over-tightening.

In emergency settings, tourniquets are applied with no readout or feedback whatsoever regarding the tourniquet pressure, meaning that it may be far in excess of the minimum required pressure, or perhaps even insufficient to provide full occlusion.

Tourniquets are also commonly used in surgical applications to reduce blood flow to an extremity. It is an effective method of improving the quality of the surgical field, affording the surgeon a bloodless area of operation. Surgical tourniquets are most often pneumatic, electrically-powered and include some form of pressure sensor. However, the exact pressure required to occlude venous and arterial blood flow beneath the tourniquet is dependent on the blood pressure of the patient, the dimensions of the anatomical site where it is applied and the characteristics of the compressed soft tissue. To allow for this uncertainty, surgical tourniquets are often applied at an arbitrary pressure of the surgeon's choosing, which is known to prevent blood flow but is also likely to be excessively high and risk damage to the underlying tissue.

The ‘gold standard’ procedure, which is typically followed when the tourniquet is correctly applied, is outlined as follows: a dedicated tourniquet must be used, since improvised devices often take too long to fabricate and their inefficiency may cause complications for the patient. The tourniquet is placed just above the wound and tightened (by a pump, windlass or some other mechanical means) until the circumferential pressure is sufficient to completely occlude arterial and venous blood flow. The time of application is recorded and submitted to the receiving hospital. It is sometimes advised that the time of application is written on the patient's forehead (or another easily visible location) to minimise the risk of the compression time going unrecorded and exceeding the recommended maximum duration of one hour. If transit time is less than an hour, the device will typically be left in place until the patient enters the operating theatre. For longer transit times and if the patient is stable, it may be possible to loosen the tourniquet as long as bleeding is fully controlled.

An example of an automatic tourniquet system can be seen e.g. in U.S. Pat. No. 5,842,996, however this device is complicated and requires substantive electric power, and it is thus not suited for emergency situations at remote locations with limited resources.

### SUMMARY OF THE INVENTION

Following the above, the inventors of the present invention have appreciated that there is considerable need for a solution which can address the aforementioned challenges faced by health workers, combat medics and the layperson in preventing haemorrhage in both surgical and resource constrained settings, while also not significantly changing their workflow. Especially, it may be seen as a problem to help the untrained user or operator in an emergency situation to be able to provide a sufficient and yet tissue safe pressure, and preferably the untrained user should be able to reach such pressure as quickly as possible.

In a first aspect, the invention provides an inflatable tourniquet system for arterial blood occlusion of a limb, the system comprising
a tourniquet arranged to be manually fastened around a limb by a user, wherein a manual inflator is connected to an inflatable chamber of the tourniquet, so as to allow application of a pressure for occlusion of arterial blood flow to the limb, upon inflation of the inflatable chamber by manually operating the manual inflator,
an electric circuit arranged to measure an electrical input from a length sensor and to determine a value indicative of circumference of the limb accordingly, when the tourniquet has been fastened around the limb,
a blood pressure measuring circuit arranged to automatically determine a measure of a systolic blood pressure (SBP) in response to input from a pressure sensor arranged to measure a pressure of the inflatable chamber,
a feedback device arranged to provide a feedback to the user, and
a processor arranged for connection to the blood pressure measuring circuit, the pressure sensor, said electric circuit, and the feedback device, wherein the processor is programmed to operate according to a control algorithm being arranged:
   to calculate a target pressure in response to the measured SBP, and said electrical resistance of the part of the conductor corresponding to a circumference of the limb,
   to monitor input from the pressure sensor and comparing a sensed pressure by the pressure sensor with the calculated target pressure, and
   to control the feedback device to provide feedback to the user, when input from the pressure sensor indicates that pressure of the inflatable chamber is within an interval of the target pressure.

Such tourniquet system is advantageous, since it provides a simple procedure to be followed by an untrained user for obtaining a pressure which is high enough to ensure arterial blood occlusion, and which is yet low enough to be safe with respect to tissue damage. This is obtained with the feedback provided to the user, e.g. light, sound, text, when the user has manually inflated the inflatable chamber to an appropriate pressure level. This is based on the insight that a target pressure can be calculated from a measured SBP and and estimated tissue padding coefficient (TPC) which can be calculated from the circumference of the limb, and that this circumference can be measured along with the user fastening the tourniquet, e.g. by measuring electric resistance in a conductor of the tourniquet, once fastened around the limb. This means that the user is not required to perform any special action to measure limb circumference. Therefore, the target pressure calculation can be performed automatically, and the procedure can be performed quickly by an untrained user, and this helps to reduce the time before the user can apply sufficient pressure to stop the bleeding. Still further, the fact that feedback is given to the user, the user will not be afraid of incidentally applying a too high pressure, and thus the user will not hesitate to perform the manual inflation in a quick manner.

Further, by the user of a manual inflator, the system only demands a small amount of electric power to drive the processor, the blood pressure meter circuit and the electric circuit used to measure resistance. Thus, this can be done by a small battery, e.g. a rechargeable battery to be powered by the manual inflator. Hereby, it is possible to provide versions of the system which are highly suited for reliable operation independent of the climatic/environmental/operational conditions or settings in which the tourniquet is deployed.

In some embodiments, to further reduce the burden on the untrained user, the system may comprise an automatic inflator or pump to inflate the inflatable chamber of the tourniquet to the target pressure, i.e. removing the need for manual inflation, after the target pressure is determined. The system could also automatically maintain this pressure for a prescribed safe amount of time. This requires an electric power source to power the automatic inflator of pump.

None of the prior art methods and system teach tourniquet application solutions which are capable of automatically calculating optimal tourniquet pressure and providing feedback to the user, whilst still being practical for deployment in emergency situations and resource constrained settings.

In essence, the tourniquet system:

1) provides use of tourniquet pressure in conjunction with automatically calculated optimal tourniquet pressure with real-time feedback to the user, assisting in the proper application of the device,
2) automatically measures limb circumference and SBP and use these parameters to calculate an optimal tourniquet pressure or target tourniquet pressure,
3) can be operated and works regardless of the conditions (weather, ambulatory/non-ambulatory, etc., climate) or settings (combat, urban, peri-urban, suburban, rural, etc.) in which it is deployed, and
4) the possibility to harvests energy for powering the tourniquet from the manual inflation action of the tourniquet.

In the following preferred features and embodiments will be described.

The processor is preferably arranged to calculate the target pressure as a sum of a first value representing the measure of systolic blood pressure using (SBP) and a second value calculated in response to said electrical resistance of said part of the conductor corresponding to a circumference of a limb. Especially, the target pressure calculated is an estimation of the Arterial Occlusion Pressure (AOP), which is the lowest pneumatic tourniquet inflation pressure required to stop arterial blood flow into the limb, and its usage has been shown to be useful in optimizing tourniquet cuff pressures. The AOP can be estimated from the SBP and the Tissue Padding Coefficient (TPC), which is based on the circumference of the limb before cuff inflation as follows:

AOP=SBP+10/TPC (1)

The Tissue Padding Coefficient (TPC) is based on the circumference of the limb before cuff inflation, and thus TPC can be estimated in response to the measured resistance of the electric conductor, e.g. by means of a prestored look-up table. Thus, the calculation of target pressure preferably involves calculating a value indicative of TPC of the limb in response to the electrical resistance of the part of the conductor corresponding to a circumference of a limb, and a value from a prestored table.

A margin for error of such as 20 mmHG may be added to the AOP yielding the following expression for target pressure, i.e. optimal AOP (OAOP):

OAOP=AOP+20 mmHG (2)

In practice, a target pressure interval can be determined based on the calculated target pressure, e.g. OAOP. The processor may be arranged to control the feedback device to provide at least three different types of feedback to the user in response to input from the pressure sensor, so as to indicate whether the pressure is: below, within, or above the calculated target pressure interval, respectively. Hereby, the user can: inflate further, stop inflation, or activate a deflation valve, respectively.

The feedback device may comprise at least a visual indicator or an audible indicator, or a combination of both. E.g. a visual indicator may provide a feedback signal by means of coloured lights, e.g. LEDs, a black white or color display (e.g., low power e-ink screen) to show text and/or symbols. E.g. an audible indicator may provide different audible tones, and/or speech to guide the user.

In preferred embodiments, the electrical conductor is mounted in a lining of the tourniquet. Especially, the electrical circuit is preferably connected to the electrical conductor, so as to allow electrical contact with respective ends of the part of the electrical conductor which corresponds to a circumference of the limb. Especially, the electric conductor is preferably arranged along the length of the tourniquet, and wherein the fastening mechanism is used to provide electric connection to a part of the electric conductor which corresponds to the circumference of the limb, thereby allowing the measuring resistance to reflect the circumference of the limb. In this way, the user only needs to fasten the tourniquet in a normal manner, and the processor can then receive a resistance value from the electric circuit, e.g. when the pressure sensor indicates that inflation has started. Especially, it is preferred that the measurement of electrical resistance is based on the principle that the resistance of a conductive wire changes with geometry, which is commonly utilized in strain gauges. The lateral and/or axial strain can therefore be correlated to the diameter of the limb. E.g., a smaller diameter limb should therefore correspond to a higher strain than a large diameter limb. For this purpose, the conductive wire is preferably integrated or built into a sleeve of the tourniquet, such that it can measure the lateral strain as it is wrapped around the limb. The electric conductor may have a zig-zag or square wave pattern of parallel lines in order to ensure that a small amount of stress in the direction of the orientation of the parallel lines results in a multiplicatively larger strain measurement over the effective length of the electric conductor.

The manual inflator preferably comprises a bulb inflator arranged for being squeezed by the user in order to inflate the inflatable chamber. In other embodiments, the manual inflator may be provided by means of a rotationally activated pump.

The system may comprise a clock arranged to determine a time of application of the tourniquet on the limb, and wherein the system is arranged to provide a feedback in response to said time of application of the tourniquet. Hereby, the E.g. the clock may be set to determine the time of application of the tourniquet on the limb to the time, where the processor receives a sensed pressure from the pressure sensor which exceeds a preset value. In a specific embodiment, a small e-ink screen and a real time clock are incorporated into the system, which together will display the time since the optimal tourniquet (or target) pressure was first achieved or alternatively a countdown from the maximum permitted tourniquet application time, e.g. one hour. This will assist medical personal in knowing the precise time of application and reduce human error arising from incorrect data recording. An alarm, buzzer or flashing light may also be added, which will be activated when the maximum application time is approached and also when the tourniquet pressure falls out of the optimal range.

In some embodiments, the system comprises an mechanical energy harvesting device arranged to generate electric energy to power at least the processor in response to manual operation of the manual inflator, e.g. all of the electric power demanding elements of the system may be powered by the mechanical energy harvesting device. Hereby, the system can function also in locations, where no electric power outlet or replacement batteries are available. Especially, the system may comprise an electric energy storage element arranged to store electric energy generated by the electric energy harvesting device. Especially, the energy harvesting device may comprise a manual inflator in the form of a bulb, where a linear alternator is used to harvest electric energy from the pumping action of the bulb, which could then be used to power some or all of the device's electronic functionality. A linear alternator can be used to directly convert the linear motion of the compression/squeezing of the bulb and handle during inflation into electrical energy. Alternatively, a rotary alternator can be used if the handle is linked via a crank or step-up gear to a rotatable flywheel (e.g., flywheel magnet rotor) connected to a dynamo with a commutator (necessary for rectification of the alternating current to direct current-since). A linear alternator may be preferred in order to make the device more compact and less bulky, however, a small rotary alternator may also be used. Alternatively, energy could be harvested from the drawing of tourniquet material through an external housing using a rotary alternator. The amount of energy stored in a (super-) capacitor could then be measured, giving information about the amount of fabric which has been drawn through the housing, thus providing a measurement of the length of tourniquet remaining around the limb and thereby indicating a measurement of limb circumference. In this way energy harvesting and measurement of a value indicative of limb circumference can be combined.

The processor may be arranged inside a casing attached to a part of the tourniquet. Alternatively, the processor may be arranged in a stand-alone device, e.g. inside a separate casing housing the blood pressure measuring circuit.

The tourniquet system of the first aspect is applicable to trained as well as untrained users to emergency or other situations where arterial occlusion of a limb is advantageous, also as part of first aid kits to be used at places without any access to electric energy sources. In particular it is envisioned that this invention will help to reduce the time of application of tourniquets, reduce the occurrence pressure-related injuries and make tourniquets more accessible to inexperienced and low-skilled users in emergency situations.

The electric circuit arranged to measure an electrical input from a length sensor and to determine a value indicative of circumference of the limb accordingly, can be implemented in various ways apart from the mentioned embodiment with measuring electrical resistance of an electric conductor.

Especially, the limb circumference may be measured using a rotating element of a known circumference placed within the an external housing of the tourniquet. This rotating element will be rotated by the tourniquet material, as it is drawn through the housing and as each full rotation is completed, a counter increases in value. If the element has a circumference of say 1 cm, 20 rotations would therefore be recorded by the counter, indicating that 20 cm of material had been drawn through the housing. The amount of fabric which has been drawn through the housing thus provides a measurement of the length of the tourniquet remaining around the limb and thereby indicates a measurement of the limb circumference.

In yet another embodiment, the limb circumference may be measured using a capacitive linear encoder, similar to those found in digital Vernier calipers. Two patterns of bars with a known separation are printed on both the tourniquet material and on the circuitry within an external housing of the tourniquet. The measured capacitance changes as the material slides through the housing, counting the number of printed bars which have passed through, thereby arriving at a measure of circumference of the limb.

In still another embodiment, the limb circumference may be measured using a capacitive measurement of tourniquet material which has been drawn through an external housing of the tourniquet. The capacitance reveals the amount of fabric which has been drawn through, which in turn reveals what length of material remains around the limb, thereby reflecting limb circumference.

In a second aspect, the invention provides a method for determining feedback to a user of an inflatable tourniquet for arterial blood pressure occlusion of a limb, the method comprising
receiving a value indicative of electrical resistance of a part of an electric conductor arranged in the tourniquet, when the tourniquet has been fastened around the limb, wherein the part of the conductor corresponds to a circumference of the limb,
receiving a value indicative of SBP determined in response to a pressure measured in an inflatable chamber of the inflatable tourniquet,
calculating a target pressure in response to the value indicative of SBP, and the electrical resistance of the part of the conductor corresponding to a circumference of the limb,
monitoring pressure of the inflatable chamber and comparing the pressure of the inflatable chamber of the tourniquet with the calculated target pressure, and
providing feedback to the user, indicating that the pressure of the inflatable chamber has reached the calculated target pressure.

In a third aspect, the invention provides a computer program product comprising computer readable program code which, when executed on a processor, causes the processor to perform the method according to the second aspect. Especially, the program code may be present on a tangible medium, e.g. a memory card or the like, a read only memory, or it may be present on a server for downloading via the internet. The processor preferably has a connected memory for performing calculations, and also a memory for storing the program code to be executed.

In general, it is appreciated that the various aspects of the invention may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 illustrates a sketch of elements of an inflatable tourniquet system, and

FIG. 2 illustrates steps of a method embodiment.

### DESCRIPTION OF EMBODIMENTS

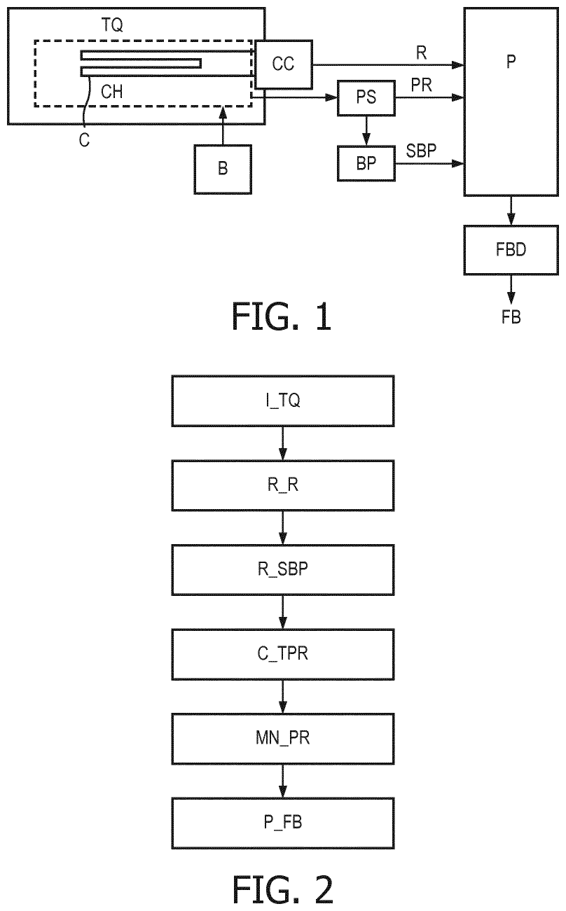

FIG. 1 shows an inflatable tourniquet system embodiment. The system is suitable for arterial blood occlusion of a limb, and it is suitable in emergency situations where even untrained users can operate the system, e.g. to provide a fast helping action to stop a bleeding from an injured limb.

The system has a tourniquet TQ arranged to be manually fastened around a limb by a user. The tourniquet TQ has a built in inflatable chamber CH in connection with a manual inflator B, e.g. a squeezable bulb, so as to allow application of a pressure for occlusion of arterial blood flow to the limb, upon inflation of the inflatable chamber by manually operating the manual inflator B. The inflatable tourniquet TQ may be similar to those known in the art with respect to its structure, size and closing mechanism etc. However, the tourniquet TQ has an electric conductor C arranged in or on its structure, which allows measurement of an electrical resistance R of a part of the electric conductor C corresponding to a circumference of a limb, when the tourniquet TQ has been fastened around the limb. The electric conductor C may be formed by e.g. copper, aluminium, steel, or other conducting material with known resistivity, preferably the electric conductor C is provided inside an insulating material. E.g. the electric conductor C is a wire. The electric conductor C is connected to an electric circuit CC which can generate a measure of the electrical resistance R of the part of the electric conductor C corresponding to a circumference of the limb. The electrical circuit CC is connected to the electrical conductor C, so as to allow electrical contact with respective ends of the part of the electrical conductor which corresponds to a circumference of the limb. Especially, the electric conductor C can be arranged along the length of the tourniquet TQ, and wherein the fastening mechanism of the tourniquet TQ (not shown) is used to provide electric connection to a part of the electric conductor C which corresponds to the circumference of the limb, thereby allowing the measuring resistance to reflect the circumference of the limb. As seen, the electric conductor C is shown to have a zig-zag or square wave pattern with conducting parts parallel with a length direction of the tourniquet, so as to indicate a preferred type of electric conductor C which allows measuring an electric resistance value varying with lateral strain as the tourniquet TQ is wrapped around the limb. In this way, the electric conductor C in accordance with the principle of a strain gauge. The electric conductor C may be mounted, e.g. integrated, in a sleeve of the tourniquet TQ.

A blood pressure measuring circuit BP is arranged to automatically determine a measure of a systolic blood pressure SBP in response to input from a pressure sensor PS arranged to measure a pressure of the inflatable chamber CH. Such automatic blood pressure measuring circuit BP is known in the art, e.g. it may operate according to an oscillometric method as known in the art. Especially, the blood pressure measuring circuit may start operating once the pressure sensor PS senses a pressure exceeding a preset value.

A feedback device FBD serves to provide a feedback FB to the user, e.g. a visible and/or audible feedback. Especially, the feedback device FBD may comprise an LED array or a similar arrangement to provide feedback FB to the user applying the tourniquet and indicate when the tourniquet pressure is above, below or within the optimal range.

A processor P, or preferably a processor system including memory etc., is arranged for connection to the blood pressure measuring circuit BP, the pressure sensor PS, the electric circuit CC, and the feedback device FBD. The processor P is programmed to operate according to a control algorithm, preferable with its executable program code stored in read-only memory. The control algorithm serves:

1) to calculate a target pressure in response to the measured SBP, and the electrical resistance R of the part of the electric conductor C corresponding to a circumference of the limb,
2) to monitor input PR from the pressure sensor PS and comparing a sensed pressure by the pressure sensor PS with the calculated target pressure, and
3) to control the feedback device FBD to provide feedback FB to the user, when input PS from the pressure sensor PS indicates that pressure of the inflatable chamber CH is within an interval of the target pressure. The target pressure is preferably calculated as in Eq. (1) or (2) as further explained below.

In this way, the user is provided with feedback FB about the manual inflation process and can thus stop further inflation, once the target pressure has been obtained.

In the following, the actual process involved in using a specific system embodiment will be described.

The manually operated tourniquet is tightened around the extremity, i.e. limb (arm or leg), by drawing the tourniquet material through an external housing until the limb is tightly encircled. The cuff of the tourniquet is then locked in place using a clamp on the tourniquet housing unit. When the manual inflator bulb is squeezed, and the inflation chamber of the cuff begins to inflate. This triggers a measurement of the limb circumference using an electric conducting wire (or wires) running through the tourniquet, and which form a loop beginning and ending at the external tourniquet housing. The electrical resistance of the loop is measured and from this the length of wire can be calculated, using known resistivity and diameter values for the wire. The processor (CPU chip) compares measured resistance with a look-up table of resistance values which correspond to a known length, e.g. 3 Ohms=30 cm limb circumference. The CPU chip then matches this limb circumference with a look-up table of known values to find the corresponding TPC.

As the tourniquet is tightened, the tourniquet is inflated manually by the user up to a pressure above SBP. An SBP measuresment can only be taken after inflation is complete, and during deflation of the inflatable chamber after a pressure above SBP has been achieved. Once SBP is determined, the tourniquet is then be inflated again to inflate it to the target pressure, i.e. preferably Arterial Occlusion Pressure (AOP) or Optimal AOP (OAOP). The target pressure is computed using Eq. (1) or Eq. (2), where TPC and SBP are used: Eq. (1): AOP=SBP+10/TPC, and Eq. (2): OAOP=AOP+20 mmHG. In OAOP, a safety margin of 20 mm HG is added. This safety margin may be chosen to be smaller or larger than 20 mmHG, e.g. it may be selected in the range 10 mmHG to 50 mmHG.

The CPU chip compares the pressure of the tourniquet with the optimal pressure and will give feedback to the user, to guide the user into bringing the tourniquet within the optimal pressure range. This is achieved, for example, using a red LED to signal that the tourniquet pressure is too low, green LED to signal that the pressure is correct and a flashing orange or blue LED to alert nearby emergency operators that the tourniquet pressure has risen above the optimal level. Alternatively, or additionally, feedback may be given to the user by means of a small screen e.g. an e-ink screen.

Once the optimal or target pressure range has been reached (for example, OAOP±5 mmHG), the user receives positive feedback from the system and will stop squeezing the bulb. Hereby the user is quickly set free to continue other treatment of the patient. SBP is preferably automatically measured at regular intervals (e.g. every 5 minutes) to check for changes in the patient's blood pressure. The calculations of AOP and OAOP are automatically repeated each time, using the original limb circumference reading for the calculation. This method allows the system to ignore changes in limb circumference caused by the pressure of the tourniquet. If the SBP has changed such that the tourniquet pressure is now outside the optimal range, feedback will once again be given to the user to adjust the tourniquet application pressure until it falls again in the optimal range.

The typical manual cuff inflation time required is estimated to be approximately 5-20 s. Based on values reported in the literature for human energy harvesting from squeezing, it is possible to estimate the power and energy harvested during cuff inflation. Assuming a gripping force of 200-250 N (roughly half of the maximum gripping force of a 30 year old male), and application of this force over a distance of 10 mm during squeezing of the bulb at a rate of 1 Hz the following, a power of such as P=±2.0-2.5 W is obtained. Hereby, the total energy harvested over the 5-20 s period may be Etot=±10-50 J.

An energy harvesting calculation can also be made for a rotary alternator, used as the tourniquet material is drawn through the external housing during the tightening around the limb before inflation. Assuming that the initial tourniquet circumference is 70 cm and the system is applied to either the arm or leg of a person 20 years or older with an average build, an energy output can be calculated to be Etotal=±22.4-53.2 J.

In conclusion, the energy available to harvest from either of the mentioned methods would likely be sufficient to record data for tourniquet pressure, limb circumference etc. and to then make calculations and provide user feedback with this data. This could be achieved using a (super-)capacitor to store energy harvested from the tourniquet application and a CPU chip and LED array for feedback as described previously.

FIG. 2 shows steps of an embodiment of a method for determining feedback to a user of an inflatable tourniquet for arterial blood pressure occlusion of a limb. The method comprises an initial step of inflating I_TQ the inflatable chamber of the tourniquet. By sensing that the pressure in the inflatable chamber has reached a preset minimum pressure value, the further steps can be initiated. This inflation is done by the user. The further steps include receiving R_R a value indicative of electrical resistance of a part of an electric conductor arranged in the tourniquet, when the tourniquet has been fastened around the limb, wherein the part of the conductor corresponds to a circumference of the limb. Further, the method comprises receiving R_SBP a value indicative of SBP determined in response to a pressure measured in an inflatable chamber of the inflatable tourniquet. Further, the method comprises calculating a target pressure C_TPR in response to the value indicative of SBP, and the electrical resistance of the part of the conductor corresponding to a circumference of the limb. Preferably, a targer pressure interval is calculated. Still further, the method comprises monitoring pressure MN_PR of the inflatable chamber and comparing the pressure of the inflatable chamber of the tourniquet with the calculated target pressure, and providing feedback P_FB to the user, indicating that the pressure of the inflatable chamber has reached the calculated target pressure.

To sum up, the invention provides an inflatable tourniquet system for arterial blood occlusion of a leg or arm, e.g. after injury or for surgery. A tourniquet TQ is to be manually fastened around the limb by a user, e.g. a first aid helper, e.g. an untrained person. A manual inflator B is used to inflatable the tourniquet to apply pressure for occlusion of arterial blood flow to the limb. An electric circuit CC measures an electrical input from a length sensor C, e.g. an electric conductor, and to determine a value R, e.g. electric resistance, indicative of circumference of the limb accordingly, when the tourniquet has been fastened around the limb. A blood pressure measuring circuit BP automatically determines a systolic blood pressure SBP in response to input from a pressure sensor PS arranged to measure a pressure PR of the tourniquet. A processor P is programmed to operate according to a control algorithm which calculates a target pressure in response to the measured SBP, and the value R indicative of circumference of the limb. Then, the processor monitors input from the pressure sensor PS and compares the sensed pressure with the calculated target pressure. Visual and/or audible feedback is give to the user, when the pressure of the tourniquet TQ is within an interval of the target pressure. In some embodiments, the manual inflator B process may be used to provide energy harvesting for electric powering the system.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An inflatable tourniquet system for arterial blood occlusion of a limb, the system comprising
a tourniquet (TQ) arranged to be manually fastened around a limb by a user,
a manual inflator (B) connected to an inflatable chamber (CH) of the tourniquet (TQ), so as to allow application of a pressure for occlusion of arterial blood flow to the limb, upon inflation of the inflatable chamber (CH) by manually operating the manual inflator (B),
a length sensor (C),
an electric circuit (CC) arranged to measure an electrical input from a length sensor (C) and to determine a value (R) indicative of circumference of the limb accordingly, when the tourniquet (TQ) has been fastened around the limb,
a pressure sensor (PS) arranged to measure a pressure (PR) of the inflatable chamber (CH),
a blood pressure measuring circuit (BP) arranged to automatically determine a measure of a systolic blood pressure (SBP) in response to input from a pressure sensor (PS),
a feedback device (FBD) arranged to provide a feedback (DB) to the user, and
a processor (P) arranged for connection to the blood pressure measuring circuit (BP), the pressure sensor (PS), said electric circuit (CC), and the feedback device (FBD), wherein the processor (P) is programmed to operate according to a control algorithm being arranged:
to calculate a target pressure (AOP, OAOP) in response to the measured SBP, and said value (R) indicative of circumference of the limb,
to monitor input from the pressure sensor (PS) and comparing a sensed pressure (PR) by the pressure sensor (PS) with the calculated target pressure (AOP, OAOP), and
to control the feedback device (FBD) to provide feedback (FB) to the user, when input (PR) from the pressure sensor (PS) indicates that pressure (PR) of the inflatable chamber (CH) is within an interval of the target pressure.

2. The inflatable tourniquet system according to claim 1, wherein the processor (P) is arranged to calculate the target pressure (AOP, OAOP) as a sum of a first value representing the measure of systolic blood pressure using (SBP) and a second value calculated in response to said value (R) indicative of circumference of the limb.

3. The inflatable tourniquet system according to claim 1, wherein the feedback device (FBD) comprises at least one of: a visual indicator, and an audible indicator.

4. The inflatable tourniquet system according to claim 1, wherein the processor (P) calculates a target pressure interval in response to the calculated target pressure.

5. The inflatable tourniquet system according to claim 4, wherein the processor (P) is arranged to control the feedback device (FBD) to provide at least three different feedbacks (FB) to the user in response to input from the pressure sensor (PS), so as to indicate whether the pressure (PR) is: below, within, or above the calculated target pressure interval, respectively.

6. The inflatable tourniquet system according to claim 1, comprising an electric conductor (C) arranged in the tourniquet (TQ), so as to allow measurement of an electrical resistance (R) of a part of the electric conductor (C) corresponding to a circumference of the limb, when the tourniquet (TQ) has been fastened around the limb, wherein the electric conductor (C) is connected to an electric circuit (CC) arranged to generate a measure of said electrical resistance (R) of said part of the electric conductor (C) corresponding to the circumference of the limb.

7. The inflatable tourniquet system according to claim 6, wherein the electrical conductor (C) is mounted in a lining or sleeve of the tourniquet (TQ).

8. The inflatable tourniquet system according to claim 1, wherein calculation of the target pressure (AOP, OAOP) involves calculating a value indicative of a tissue padding coefficient of the limb in response to the value (R) indicative of circumference of the limb, and a value from a prestored table.

9. The inflatable tourniquet system according to claim 1, wherein the manual inflator (B) comprises a bulb inflator (B) arranged for being squeezed by the user in order to inflate the inflatable chamber (CH).

10. The inflatable tourniquet system according to claim 1, comprising a clock arranged to determine a time of application of the tourniquet (TQ) on the limb, and wherein the system is arranged to provide a feedback in response to said time of application of the tourniquet (TQ).

11. The inflatable tourniquet system according to claim 1, comprising an electric energy harvesting device arranged to generate electric energy to power at least the processor (P) in response to manual operation of the manual inflator (B).

12. The inflatable tourniquet system according to claim 11, comprising an electric energy storage element arranged to store electric energy generated by the electric energy harvesting device.

13. The inflatable tourniquet system according to claim 1, wherein the processor (P) is arranged inside a casing attached to a part of the tourniquet (TQ).

14. A method for determining feedback to a user of an inflatable tourniquet for arterial blood pressure occlusion of a limb, the method comprising
receiving (R_R) a value indicative of a circumference of the limb (R),
receiving (R_SBP) a value indicative of systolic blood pressure (SBP) determined in response to a pressure measured in an inflatable chamber of the inflatable tourniquet,
calculating (C_TPR) a target pressure in response to the value indicative of systolic blood pressure (SBP), and the value indicative of a circumference of the limb (R),
monitoring (MN_PR) pressure of the inflatable chamber and comparing the pressure of the inflatable chamber of the tourniquet with the calculated target pressure, and
providing (P_FB) feedback to the user, indicating that the pressure of the inflatable chamber has reached the calculated target pressure.

15. A computer program product comprising computer readable program code which, when executed on a processor, causes the processor (P) to perform the method according to claim 14.
